(19) United States
(12) Patent Application Publication
(10) Pub. No.: US 2026/0360546 A1
(43) Pub. Date: Nov. 12, 2026

(54) Distributed Multi-Modal Systems and Methods for Monitoring and Determining the Condition and Performance of Personnel and Teams

(71) Applicant: Hatch Systems, Inc., Dover, DE (US)

(72) Inventors: Berenice Charrez, San Francisco, CA (US); Nicolas Antonsen, Berlin (DE)

(73) Assignee: Hatch Systems, Inc., Dover, DE (US)

(21) Appl. No.: 19/673,824

(22) Filed: May 11, 2026

Related U.S. Application Data
(60) Provisional application No. 63/803,734, filed on May 11, 2025.

(51) Int. Cl. G16H 50/20; G06F 3/01

(52) U.S. Cl. CPC G16H 50/20; G06F 3/011

(57) ABSTRACT

Disclosed herein are systems and methods for monitoring and determining the condition and performance of individuals and groups of individuals. The systems and methods are distributed and multi-modal, meaning that the systems and methods are arranged to gather data on individuals and groups of individuals where the individuals and/or physical components of the systems are geographically distributed and the monitoring and determination of conditions and performance is accomplished by gathering measurements for a number of different aspects of the individuals and/or the environment surrounding the individuals and analyzing the data generated or inferred by the measurements. The system can further analyze the data to predict the future conditions of individuals, their readiness to begin and/or complete a task, and render recommendations to individuals and those responsible for the individuals regarding beginning and/or completing tasks.

### CROSS REFERENCE TO RELATED APPLICATIONS

This application claims priority from U.S. Provisional Patent Application No. 63/803,734, filed on May 11, 2025, which is incorporated by reference herein in its entirety.

### FIELD OF DISCLOSURE

Generally, the present disclosure is directed to systems and methods for monitoring the condition and performance of individuals and teams of individuals. More specifically, the present disclosure is directed to gathering physiological and cognitive data about individuals, gathering data about the environment surrounding the individuals; gathering additional data regarding forecasted changes in the environment surrounding the individual; determining the physical and mental condition of the individuals and/or teams; predicting future conditions of the individuals or team based on gathered data; and providing feedback and making actionable recommendations to the individuals and/or teams.

### BACKGROUND

In all endeavors, whether professional, recreational, or everyday tasks, individuals seek to perform at an adequate level and ideally perform at their peak physical and mental performance. This is also true for teams of individuals seeking to collectively reach their peak performance level. An individual’s or team’s ability to accomplish tasks is often dependent on the physical and mental condition of the individuals as well as the environmental conditions surrounding the individuals. In certain professional and athletic activities, an individual’s ability to accomplish a task is critical to not only completing a task successfully, but also to ensure public safety and the safety of teammates. In one example, firefighters often work for long hours under grueling conditions that challenge the firefighter’s physical and mental endurance. It is often left to the firefighter’s own judgment to determine if the firefighter is in a condition to continue fighting the fire or whether the firefighter should rest and recover for a period of time. Relying on such ad hoc decisions made by personnel that are under stress and potentially mentally and physically fatigued can lead to poor decision making that jeopardizes the health and safety of individuals and team members working with the individuals.

There are currently a number of wearable devices that can monitor certain aspects of an individual’s physical condition. However, wearables by themselves do not provide sufficient information to improve decision making. Therefore, there is a need for novel comprehensive systems and methods that: i gather data, in real time, about several aspects of an individual’s or group of individuals’ physical and mental condition along with surrounding environmental conditions; (ii) analyze the gathered data and translate the data into quantifiable measures of an individual’s or team of individuals’ real-time condition and performance; (iii) provide predictions of future condition and performance of the individual or group of individuals; and iv render actionable recommendations for the individual or group of individuals, where such systems and methods can operate and are rendered locally regardless of location or access to communication networks. Such novel systems and methods are disclosed and described herein.

### SUMMARY

Disclosed herein are systems and methods for monitoring and determining the condition and performance of individuals and groups of individuals (teams). The systems and methods are distributed and multi-modal, meaning that the systems and methods are arranged to gather data on individuals and teams where the individuals and/or physical components of the systems are geographically distributed and the monitoring and determination of conditions and performance is accomplished by gathering measurements for a number of different aspects of the individuals and/or the environment surrounding the individuals.

In one embodiment, the system includes at least one wearable device secured to an individual, at least one environmental device positioned in the environment surrounding the individual; and a central hub spaced away from the individual. The wearable device is arranged to measure at least one physiological or cognitive attribute of the individual and to transmit that data to the central hub. The environmental device is arranged to measure at least one aspect of the environment surrounding the individual and to transmit that data to the central hub. The central hub is arranged to analyze the gathered data, make determinations on the current condition of the individual, make a prediction for a future condition of the individual; and notify the individual or other applicable person of current condition and predicted future condition of the individual. The system can be further arranged to gather additional data that can also be analyzed to determine the current condition and predicted future condition of the individuals and/or teams. Examples of such additional data include forecasted near-term changes in the environment surrounding the individual (e.g., weather reports and forecasts) and positional data (e.g., global positioning system (GPS) data).

In another embodiment similar to the embodiment described above, the individual can be further equipped with a portable hub. In this embodiment, at least one wearable device and at least one environmental device or sensor transmit data to the portable hub, and the portable hub is arranged to perform some or all of the analysis on the gathered data and actions described above. In an embodiment where the portable hub performs some of the analysis on the gathered data and further actions, the portable hub can be arranged to transmit data and other information to the central hub for further analysis and processing. In an embodiment where the portable hub performs all of the analysis on the gathered data and further actions, the portable hub can fully replace the central hub. In certain embodiments, the environmental device can be an environmental sensor integrated into the portable hub.

Multiple central hubs and portable hubs can be arranged to communicate together through a mesh network via radio frequencies (e.g., LoRaWAN). Where the system further includes remote computing functionality (arranged as a platform) that is cloud based or resides at a remote data center, the one or more central hubs and one or more portable hubs can be arranged to transmit and otherwise upload data to such a platform. Such transmissions can be made via a variety of technologies such as 4G, LTE, satellite, and WIFI.

In one embodiment, a method includes the steps of: gathering data regarding an individual’s physiological and/or cognitive conditions; gathering data regarding the environment surrounding the individuals; gathering data regarding near-term changes to the environment surrounding the individuals; analyzing the gathered data; making a determination of the current physiological and cognitive condition of the individual; predicting the future physiological and cognitive condition of the individual; generating a recommendation regarding the individual; and relaying that recommendation to the individual or other person responsible for the individual.

In other embodiments, the central hub, portable hub, or other such computing devices used with a system or method can include actionable protocols. Such protocols trigger actions in response to the gathered data or the analysis of the gathered data, such as, for example, alarms or recommendations, based on the measured, inferred, or the forecasted condition of the individual, the team, or the surrounding conditions.

### BRIEF DESCRIPTION OF THE DRAWINGS

In the accompanying drawings, structures are illustrated that, together with the detailed description provided below, describe example embodiments of the disclosed systems, methods, and apparatus. Where appropriate, like elements are identified with the same or similar reference numerals. Elements shown as a single component can be replaced with multiple components. Elements shown as multiple components can be replaced with a single component. The drawings may not be to scale. The proportion of certain elements may be exaggerated for the purpose of illustration.

FIG. 1 schematically illustrates an individual equipped with a wearable device.

FIG. 2 schematically illustrates an embodiment central hub.

FIG. 3 schematically illustrates another embodiment of a central hub.

FIG. 4 schematically illustrates an individual equipped with a wearable device and a portable hub with integrated environmental sensors.

FIG. 5 schematically illustrates a firefighter equipped with a wearable device and a portable hub, with environmental sensors integrated into the portable hub.

FIG. 6 schematically illustrates a team including firefighters and a supervisor using a exemplary system.

FIG. 7 schematically illustrates a runner equipped with a wearable device and a portable hub.

FIG. 8 schematically illustrates a report charting physiological and environmental conditions over time.

FIG. 9 schematically illustrates a system where wearable devices communicate directly with a central hub that includes integrated environmental sensors.

FIG. 10 schematically illustrates a dashboard displaying environmental conditions, physiological conditions, and status of individuals.

FIG. 11 schematically illustrates an exemplary system diagram.

### DETAILED DESCRIPTION

Disclosed herein are systems and methods generally arranged to monitor individuals or groups of individuals (teams) and make determinations and/or recommendations regarding the individuals and/or teams. More specifically, the systems and methods are arranged to perform one or more of the following actions: gather data regarding individuals’ physiological and/or cognitive conditions; gather data regarding the environment surrounding the individuals; gather data regarding near-term changes to the environment surrounding the individuals; gather other data applicable to the individuals and/or their surroundings; analyze the gathered data; make a determination of the current physiological and cognitive condition of the individuals; predict the future physiological and cognitive condition of the individual based on gathered data; generate a recommendation regarding the individuals; and relay those recommendations to the individuals or other persons responsible for the individuals. As will be further described, the system can include a variety of components such as monitoring devices, sensors, local computing devices, cloud-based computing devices, remote computing devices, and hardware and functionality to facilitate communications between and among such components. In certain embodiments of the system and methods, data can be transmitted between and/or among components of the system using peer-to-peer network arrangements, mesh networking arrangements, client-server arrangements, and other such technologies.

The systems and methods described herein are useful in assisting individuals or teams in reaching peak physical performance and, in certain circumstances, are useful in ranking or prioritizing individuals based on current or predicted physical and mental conditions. Generally, the systems and methods can be implemented using edge computing systems and edge hardware, meaning that the processing of data occurs in close proximity to where the data is generated and gathered. In certain embodiments, critical physiological, environmental, operational, and alert data can be transmitted between distributed edge hubs over long distances using radio-frequency communication systems, thereby enabling team-level monitoring, prioritization, coordination, determinations, and recommendations to be generated at locations significantly distant from where the underlying data is acquired. In certain embodiments of the systems and methods, functionality, such as the storage of data, analysis of data, training of models, and distribution of determinations and recommendations, can occur on or with the assistance of remote servers located in a centralized cloud or data center.

For the sake of conciseness, the system and methods disclosed herein can be referred to collectively simply as a “system” or “systems.” Any description of how the systems apply to an individual can equally be applied to a team of individuals. The term “condition” of an individual as used herein can include both the individual’s physiological condition and cognitive condition.

The system can be arranged to gather data about an individual’s physiological and/or cognitive conditions through one or more devices, including wearable devices, arranged to measure one or more aspects of an individual’s conditions. The term measure or measured as used herein refers to acquiring data representative of a given aspect or condition of an individual or environment. For example, aspects of an individual’s physiological measurements can be gathered using photoplethysmogram (PPG) devices, electrocardiogram (ECG) devices, galvanic skin response (GSR) devices, temperature devices, glucose sensor and other metabolic devices, and the like. An individual can be fitted with wrist-based, arm-based, finger-based, head-based, ear-based, patch-base and/or chest-based wearable devices. For cognitive measurements, an individual can be equipped with a portable electroencephalogram (EEG) device or another similar device. Additional devices such as accelerometers, gyroscopes, and GPS enabled devices can be used to gather data on an individual’s geographic position and movements over time. It will be understood that any device that can make measurements of an individual based on contact with the individual, proximity to the individual, or movement of the individual can be used with the system. Such devices can be referred to herein as wearable devices.

The type of conditions measured for individuals by wearable devices include heart rate, heart rate variability (HRV), blood oxygen saturation levels, body temperature, hydration levels, blood pressure, electrical activity of the brain, and the like. Additionally, the system can measure movement of the individual over time and the current location of the individual. It will be appreciated that a 6-axis accelerometer or gyroscope and motion sensing device can be used to measure quick movements of an individual’s head, which can be used to determine impacts to the head and ultimately risks of concussions.

The system can be arranged to gather data regarding the environment surrounding the individuals through one or more devices including sensors. For example, the system can use digital thermometers, hygrometers, light sensors, noise sensors, particulate, chemical, and radiation sensors, and the like. Such devices and sensors can be used to gather data on ambient temperature, humidity, rainfall, wind level, particulate count of airborne substances, ambient noise, the presence of dangerous conditions such as chemicals or radiation, barometric pressure, sound levels and pressure, and ambient light. Such devices and sensors can be referred to herein as environmental devices. The system can be arranged to gather data regarding near-term or long-term changes to the environment surrounding the individuals. For example, the system can be arranged to continuously or periodically receive weather forecast data from a weather service such as, for example, the National Weather Service (NWS) and National Oceanic and Atmospheric Administration (NOAA), operated by the United States government, European Centre for Medium-Range Weather Forecasts (ECMWF), Global Forecast System (GFS), operated by European governments, and other such national or reginal governmental weather services. The system can further be arranged to receive forecast data from any number of private or commercial organizations. Such weather forecast information can be used by the system to predict the environmental conditions at a future time, whether in the near-term (i.e., what will the temperature, humidity, rainfall, etc. be in one hour) or the long-term (i.e., what will environmental conditions be next week, next month, etc.).

The system can be arranged to gather other data applicable to the individuals and their surroundings. For example, the system can gather information from motor vehicles, aircraft, or watercraft on which individuals are traveling. In one example, if individuals are traveling on a boat, the system can gather the speed of the boat, wind angle, wind speed, sea state (water roughness, wave height and characteristics), and the like. Such data can be used to calculate or infer physiological responses such as exertion level, fatigue, recovery efficacy, stress levels, environmental strain, cognitive readiness, performance capacity, injury risk, and other indicators associated with the condition or operational status of the individuals. In another example, if the individual is traveling by plane, the system can gather plane speed, change in direction, external temperature, and the like. In one example, such data can be used to calculate or infer g-forces experienced by individuals traveling in the plane.

The system can be arranged to analyze the gathered data through one or more algorithms arranged to use physiological, cognitive, and/or environmental measurements as inputs and perform analysis on such inputs. It will be appreciated the system can be arranged to analyze data to infer a condition. In one example, the algorithm can include functionality that analyzes factors such as an individuals’ body temperature, heart rate, respiratory rate, and perspiration rate along with environmental conditions such as ambient temperature and humidity to infer whether or not an individual is at risk of suffering heat stroke.

The system can include a central computing device, referred to herein as a central hub that is generally spaced away from the monitored individuals (meaning the central hub is not located on or does not travel with the individual as the individual moves). The central hub can be located a short distance away from the individuals or may be located a large distance away from the individuals. In certain embodiments, the central hub can include integrated environmental sensors and can directly communicate with wearable devices located within wireless communication range of the central hub, such as Bluetooth®/ANT+ communication range. Such an arrangement can be advantageous in operational environments such as sailboats, vehicles, aircraft, medical facilities, stadium sports, or other environments where individuals remain in relatively close proximity to the central hub.

Additionally, or alternatively, the system can include a portable hub that is generally attached to the individual and can assist or replace the central hub. In one embodiment of the system, the central hub and/or portable hub includes algorithms and other software useful to the system. Generally, the central hub is located remotely from the wearable devices, while the portable hub can be located very close to the wearable devices (as both the portable hub and wearables can be attached to the individual). In this embodiment, the wearable devices include structure and functionality to transmit data to the central hub and/or portable hub and optionally, structure and functionality to receive data from the central hub and/or portable hub. Reciprocally, the central hub and/or portable hub includes structure and functionality to receive data from the wearable devices, and optionally, transmit data to the wearable devices.

In this embodiment, the central hub and/or portable hub can include the environmental devices, meaning the environmental devices are integrated with the central hub or portable hub, or the environmental devices can be independently positioned in the environment surrounding the individuals. When environmental devices are integrated with a central hub or portable hub, they can be referred to as environmental sensors. The positioning of the environmental devices or sensors can be determined by the specific use of the system. For example, if the individuals are located near the central hub during the monitoring of the individuals or if the environmental conditions surrounding the individuals is the same or similar to the environmental conditions at the central hub, the environmental devices can be included in the central hub as environmental sensors. If the environmental conditions surrounding the individuals are materially different than the environmental conditions surrounding the central hub, then the environmental devices can be located in the environment surrounding the individuals, proximate to the individuals, or integrated directly in the portable hub as sensors. The portable hubs include structure and functionality to transmit data to the central hub and optionally, structure and functionality to receive data from the central hub. Such transmission of data can be facilitated by a mesh network via radio frequency. If the environmental devices are independent from the central hub or portable hub, they will include structure and functionality to receive and send data to either central hubs or portable hubs. Likewise, the central hub and/or portable hub include structure and functionality to receive data from the environmental devices and optionally, includes structure and functionality to transmit data to the environmental devices.

In another embodiment, the wearable devices themselves can include algorithms and other software useful to the system. The portable hub and/or central hubs include structure and functionality to transmit data to the wearable device, and the wearable devices include structure and functionality to receive data from the portable hub and/or central hubs. The wearable device includes the components necessary to analyze data and make determinations and recommendations regarding the individual. In this embodiment, as well as others, the environmental devices can be attached to the individual in a manner in which the individual does not affect the measurements of the environmental device. For example, if the environmental device is arranged to measure ambient temperature, the environmental device can be positioned away from the individual’s body, such as on the outside of the individual’s garments, so that the individual’s body heat does not affect the ambient temperature measurement. Generally, if environmental devices are integrated in the portable hub, the portable hub will be positioned on the outside of the individual’s garments.

The system can be arranged to apply the analysis of the algorithms to make a determination of the current physiological and cognitive condition of the individual. For example, the algorithm can include or dynamically calculate acceptable ranges for certain physiological and cognitive measurements or a combination of measurements of individuals. These ranges can be normalized for all individuals or particularized for the specific individual. In one example, an acceptable range for heart rate across all individuals at rest can be set to 50-70 beats per minute and a range of 80-140 beats per minute for vigorous activities. In this example, the algorithm can use a number of conditions to determine if the individual is at rest or engaged in vigorous activity such as body temperature, perspiration rate, and movement of the individual over time. If it is determined that the individual is at rest, the algorithm makes a determination that the individual has a heart rate out of acceptable range if it is higher than 70 beats per minute. If it is determined that the individual is engaged in vigorous activity, the algorithm makes a determination that the individual has a heart rate out of acceptable range if it is higher than 140 beats per minute. The algorithm can take into account environmental conditions as well. For example, the algorithm can adjust its handling of perspiration rate based on ambient temperature by requiring a higher perspiration rate for higher ambient temperatures when determining if a person is at rest or is engaged in vigorous activity. It will be understood that the algorithm can also set acceptable ranges per an individual. The algorithm can take into account an individual age, height, weight, body fat percentage, and the like in setting acceptable ranges. The algorithm can also be trained by taking an individual’s past measurements or any training data generated by the individual into account when setting acceptable ranges.

Similarly, the system can be arranged to apply the analysis of the algorithms to predict future physiological and cognitive conditions of the individuals or the team. Once the current condition of the individual is determined and any trends in change of that condition are taken into account, the algorithm can further analyze the environmental measurement to predict how the individual’s condition will change over time due to those environmental conditions. The algorithms can take into consideration past physiological information, such as accumulated fatigue, duration of activity, and other such factors to make predictions regarding the individual’s future condition. Furthermore, the algorithm can use external data such as weather forecasts, which provide anticipated temperature, rain, and the like, effects of the time of day, and the like, as an input to predict the individual’s future condition.

The system can further be arranged to apply the algorithm to generate a recommendation regarding the individual. For example, if the algorithm determines that one or more of individual’s conditions is outside the acceptable range, the algorithm can generate a recommendation that the individual cease current activity or alternatively generate a warning that one or more of individual’s conditions are outside the acceptable range and the individual should decrease the intensity of the activity. In another example, if the algorithm determines that all individual’s conditions are within acceptable ranges, the algorithm can generate a recommendation that the individual can continue the current activity or even increase the intensity of the activity.

In another embodiment, the algorithm can be arranged to prioritize individuals based on the severity of the individual’s condition. For example, in a triage situation where medical personnel need to make quick decisions on what patients need immediate attention, the algorithm can provide a prioritized list of patients from most critical to least critical to direct or assist medical personnel in making such decisions.

The system can be arranged to relay the recommendation generated by the algorithm to the individual or other person responsible for the individual. In one example, the central hub and/or portable hub can be arranged to alert the individual to the recommendation. In one embodiment, the portable hub can include a single multi-modal light or multiple lights where a green light indicates it is safe to continue activity; an orange light indicates it is safe to continue, but proceed with caution; and a red light indicates that the individual should cease the activity. In another embodiment, such an alert system can be incorporated into the wearable device and the central and/or portable hub transmit data to the wearable device to facilitate the recommendations. This is but one embodiment, and it will be understood that the recommendations can be relayed in any number of ways including, but not limited to, audio alerts, vibration alerts, text messages, and the like. In another embodiment, a person responsible for individuals, such as a supervisor, can receive messages regarding recommendations for each individual on a team.

The system can gather data over time and further use data from other sources, such as written or dictated notes, defined warning protocols, and videos or vehicle extracted information (for example vehicle speed, wind information), useful in correlating physiological, cognitive, and environmental measurements to individual’s conditions. Such data in combination with acquired physiological and environmental information can be textualized and stored in a database or other such repository and used as training data for algorithms. The system can include one or more artificial intelligence (AI) models or agents to assist the algorithm to analyze inputs and generate one or more outputs based on learned relationships within training data. The system can further utilize a large language model (LLM) trained on the textualized data to generate, classify, summarize, predict, or otherwise process natural language content useful to the system. The system may utilize supervised learning, unsupervised learning, reinforcement learning, deep learning, neural networks, probabilistic models, statistical models, or combinations thereof.

The systems are beneficial for facilitating peak human performance and safeguarding the safety and wellbeing of individuals and teams. As will be appreciated, the systems and methods described herein are applicable to a wide range of activities, including professional activities, sports activities, and everyday activities for individuals with medical concerns. The system is particularly applicable to activities conducted in dangerous, stressful, or harsh environments. Such environments expose individuals and teams to health and safety risks and even risk of death. The algorithms can be arranged to analyze gathered information, compare that gathered data to the dangers provided by a specific task or activity, and make a determination on the readiness of an individual or team to engage in and complete the task or activity. As used herein, the term “readiness” means an individual’s or team’s ability to engage in and successfully complete a defined task without an unacceptable risk to the individual’s or team’s physical and mental health and wellbeing.

A few examples of applicable uses of the systems disclosed herein include professionals such as first responders, search and rescue personnel, fighter pilots, airline pilots, military personnel, construction workers, transportation workers, miners, oil and gas workers, electrical workers, surgeons, and other medical personnel; athletes, such as long distance runners, cross country skiers, sailing athletes, and endurance sports athletes; and individuals with medical conditions such as heart attack or stroke risk engaging in common physical activity such as exercise, household chores, and gardening. While certain aspects and embodiments will be described herein using examples specific to a given profession or activity, it will be understood that the systems and methods are applicable to any set of circumstances where an individual or team benefits from a determination of whether it is safe and/or effective to begin or continue a defined task.

In further description of the system, the system can make determinations regarding: an individual’s real-time performance in conducting an activity, whether it is dangerous for the individual to continue the activity (based on the measurements related to the individual, measurements related to the environment, or both), and whether it is likely that the individual can complete the task. The system can further provide real-time alerts to the individual or a person responsible for the individual relaying the determinations to either reassure the individual that it is safe to continue the activity or warn the individual to cease the activity. The above-described methods can be applied to a team as well. It will be understood that even if the majority or nearly all individuals on a team are performing well and a determination is made that such performance will continue, there are situations where if one team member’s performance can fall below an acceptable level, all team members may be exposed to danger. In such a situation, the systems and methods can send alerts to all team members to cease the activity.

As the individual’s measurements and environmental measurements are gathered, the system can dynamically process and analyze the data in real-time to determine if an individual’s physical and cognitive condition is sufficient for the individual to continue to perform the task in the environmental conditions. In essence, the measurements can be compared to general preset limits, or limits tailored to the individual, to determine if the individual is in physical or mental danger, is likely to see a significant decrease in performance, or if the individual can continue on without risk of physical or mental harm performing the task within an acceptable range. The individual is continuously or periodically alerted of the determination and is advised to either continue to task, proceed with caution, or to cease the task. As previously described, the systems can similarly gather data and information on several individuals in a team to determine the team’s fitness to continue a task. It will be appreciated that certain tasks require significant coordination and if one or more individual’s performance is suboptimal, all members of the team can be in jeopardy.

While the systems disclosed herein are often directed to monitoring the performance of individuals engaged in a task, the system can also be useful in generally determining the condition of an individual or a group of similarly situated individuals. In one example, medical personal managing a mass casualty event such as an accident, natural disaster, or military operation can benefit from the system. In such an example, wearable devices can be attached to each patient as the patient arrives at the triage location. The wearable device can continuously monitor vital signs of the patient such as heart rate, blood pressure, respiratory rate, and the like as well as gather environmental data. The wearable device can transmit this data to a central hub, where an algorithm can assess the critical nature of the patient’s condition and compare it to all other patients, and provide a prioritized list of patients to guide the medical personnel in treating the patients. It will be appreciated that the described system can alleviate the need for medical personnel to take periodic measurements of vital signs of multiple patients and provides direction to medical personnel to provide medical treatment in a timely manner to the most critical of patients.

For transmissions of data between the wearable devices, environmental devices, central hubs, and/or portable hubs, such transmission can be accomplished through wireless techniques. In one example, data can be transmitted over short or long distances using Bluetooth®/ANT+ protocols between wearable devices and central hubs and/or portable hubs. In another example, data can be transmitted over long distances via radio-frequency communication protocols between portable hubs and a central hub, thereby enabling distributed or meshed communication architectures in which multiple portable hubs and/or central hubs communicate with one another and support remote monitoring and coordination by team leaders or other operators. While systems can use communication methods such as cellular, satellite, and WIFI, communications do not rely on cellular, satellite, or WIFI connectivity to communicate. This is to say that systems are arranged as edge-first systems that gather physiological, cognitive, and environmental data from one or more wearable devices and environmental devices and perform localized analysis on the data on the hubs. This allows the systems to perform required tasks regardless of the availability of third-party communication equipment and services. Systems are equipped with all hardware, software, and methods required to perform required tasks locally without reliance on any hardware, software, or services outside of the system.

The central hub is arranged as a robust system that can continuously operate in harsh or extreme environments. The central hub can be arranged with its own power supply or can be powered by external batteries or vehicle, plane, or boat power systems. Dual Bluetooth® and/or ANT+ technology can be used to simultaneous manage multiple wearable devices with adaptive assignment based on signal quality.

In one embodiment, the portable hub is arranged as a compact and ruggedized system configured for operation in harsh or extreme environments and for deployment directly with individual users or mobile teams. The portable hub can include an integrated battery power supply configured to provide extended operational autonomy over prolonged periods without external charging or infrastructure support. In certain embodiments, the portable hub can communicate with wearable devices, environmental sensing devices, other portable hubs, and/or central hubs using Bluetooth®, ANT+, radio-frequency communication protocols, or combinations thereof.

As described, the central hub, portable hub, and its embedded software can make determinations in real-time and provide actional feedback directly to individuals or those responsible for the individuals during the activity. An exemplary method incudes the steps of: (i) transforming raw data from wearable devices and environmental devices into interpretable scores (e.g. heat strain, vigilance levels, environmental safety, and the like); (ii) normalize physiological, cognitive, and environmental data on a scale from “safe to continue” to “critical danger, must stop” or for triage applications, from “stable” to “needs immediate attention,” based on each individual’s baseline metrics; and (iii) relay actionable feedback to individuals or those responsible for the individuals. In addition to the color-coded light display described above, the feedback and alert functionality can be arranged in a variety of ways such as on a dashboard, rendered on a computing device, or other similar methods.

In order to more efficiently and effectively determine whether individuals should continue or cease an activity, the system and methods can include a baseline fitness level for each individual. In one example, an individual can perform a series of training exercises where he or she conducts activities of varying difficulty in varying environmental conditions. Based on the physiological and cognitive measurements during these training exercises, a baseline fitness level can be established for the individual for reference in determining when an individual is in physical danger during future activities.

Data collected during activities can be stored and added to prior data to continuously train and develop advanced and long-term individual and team strategy optimization algorithms. The data collected and the algorithms developed can provide insight and recommendations for individual and team strategy including, but not limited to, rotation optimization, nutrition and hydration planning, resource allocation, sleep requirements, resting schedules, physical training, and recovery planning. Such activities can be performed in secondary systems and need not be performed by the central hub, although the central hub can be arranged to perform such activities.

Insights for long term strategy are made actionable with the following techniques: (i) uploading auxiliary data such as, but not limited to, GPS tracking, video recordings including body camera recordings, activity notes or pictures, and voice recordings to a platform residing on a server either locally, at a cloud based location, or a data center; (ii) administering surveys to capture psychological and mental strain and perceived exertion for early trauma detection; and (iii) applying machine learning algorithms to correlate mental load, environmental stress, and physiological responses to inform predictive algorithms during activities, and provide a holistic understanding of human response in those environments.

The uploaded auxiliary data can be overlaid with the physiological, cognitive, and environmental data from activities and inform strategic debriefing and triage sessions held by team leaders to improve the overall performance of the team and the algorithms. Teams have access to surveys to assess the mental and psychological strain and perceived exertion for early trauma detection to add more data to the system and methods. Surveys can be filled out as often as needed and bring in a qualitative assessment of mental health together with quantitative biological and environmental data to inform long term mental awareness strategies. Advanced algorithms are arranged to show the correlation between mental load, environmental condition, and physical responses to inform predictive algorithms during activities and provide a holistic understanding of human response in those environments.

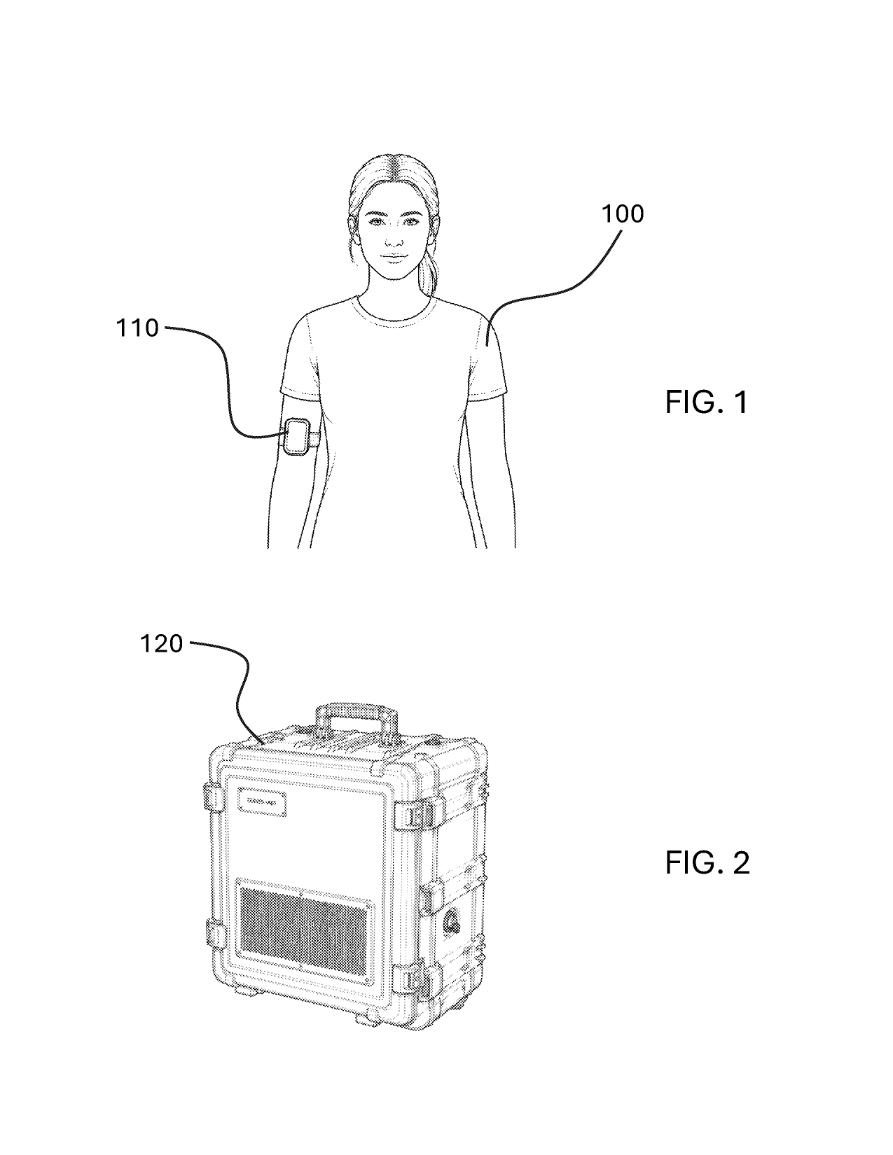

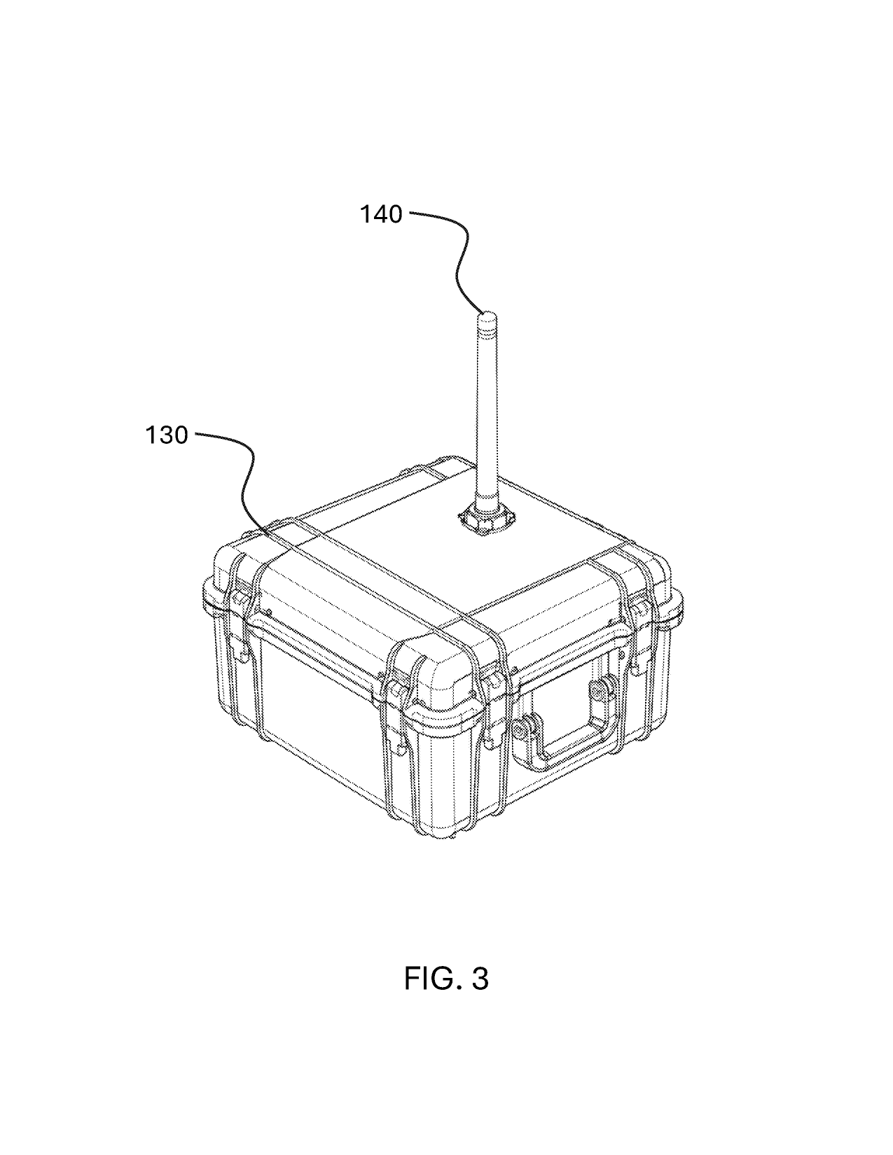

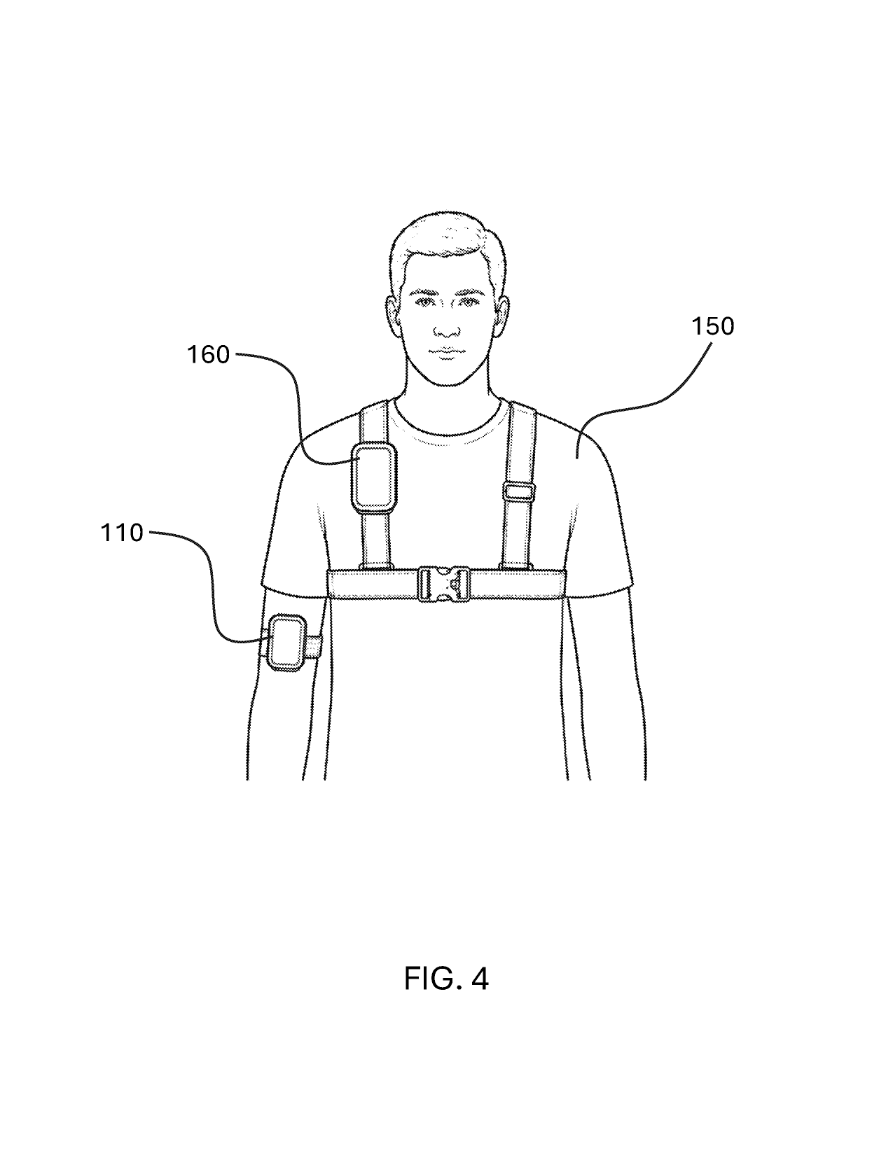

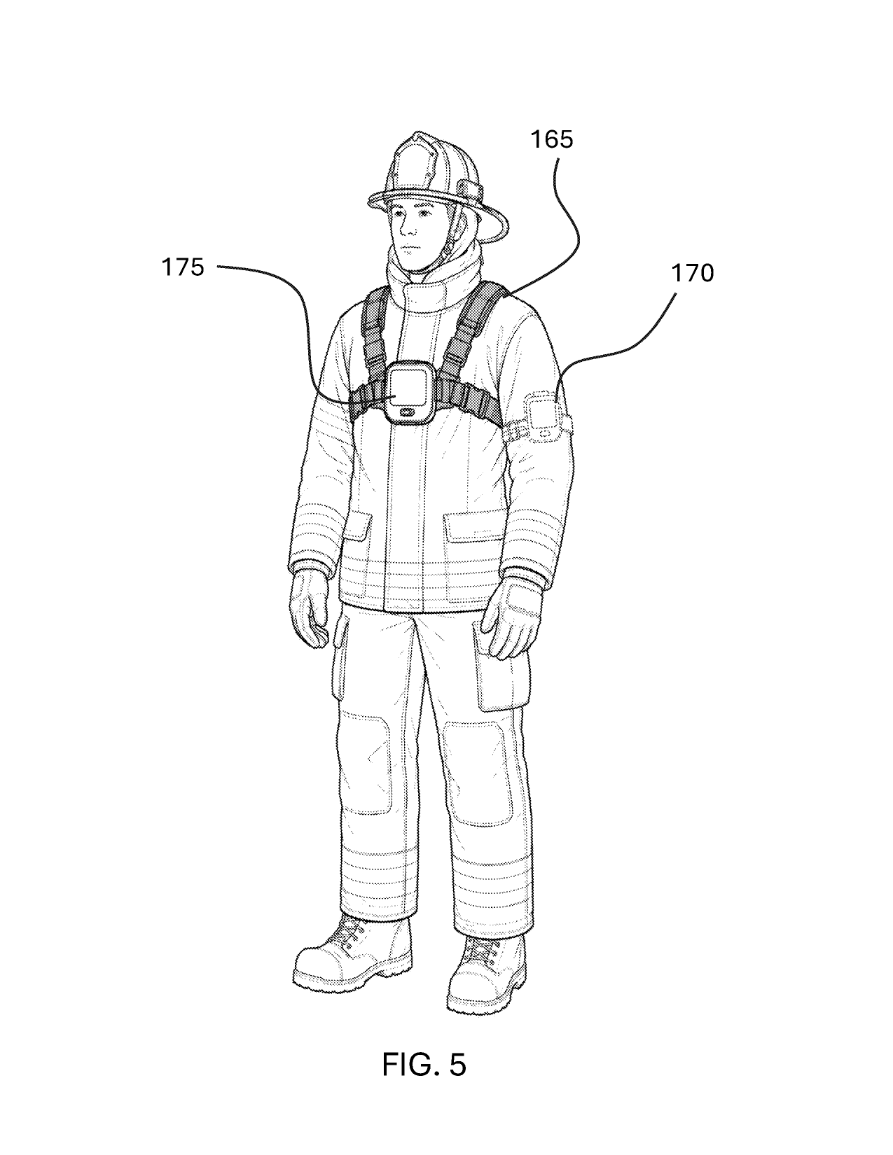

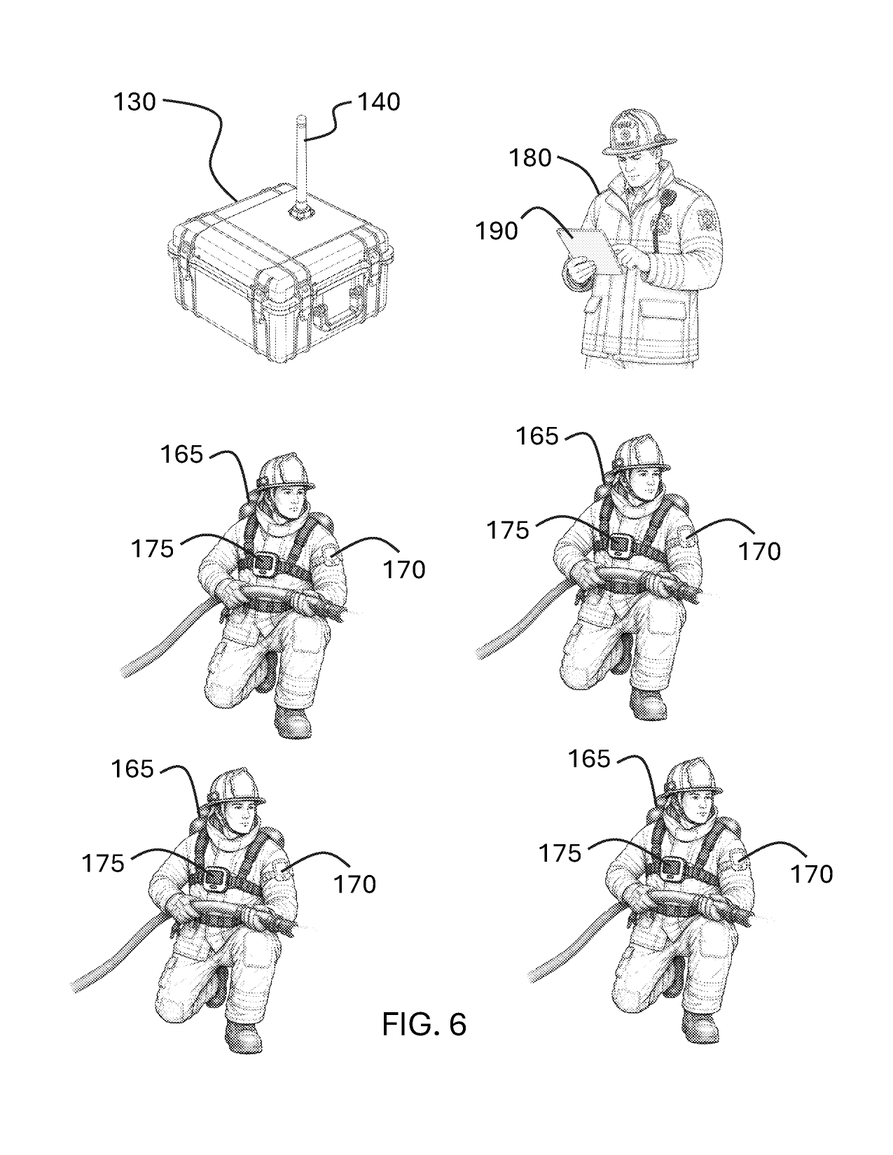

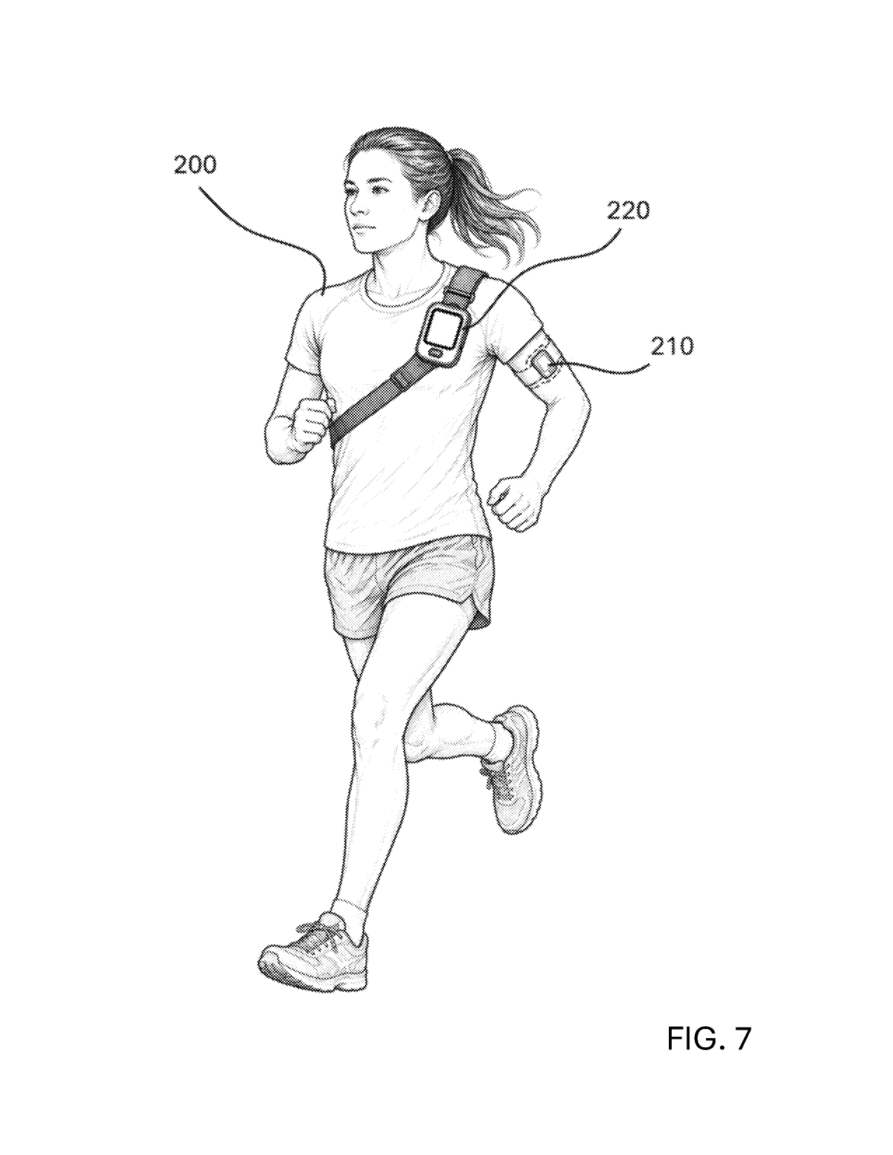

A number of embodiments will now be described with reference to the drawings. FIG. 1 schematically illustrates an individual 100 equipped with a wearable device 110. The wearable device 110 can be wrist-based, arm-based, finger-based, head-based, ear-based, patch-based and/or chest-based. It will be understood that while only one wearable device is illustrated, the individual 100 can be equipped with multiple wearable devices. FIGS. 2 and 3 schematically illustrate embodiments of central hubs 120 and 130. FIG. 4 illustrates an individual 150 equipped with a wearable device 110 and a portable hub 160. FIG. 5 illustrates a firefighter 165 equipped with a wearable device 170 (shown in dashed lines) located under the firefighter’s 165 protective clothing and a portable hub 175 with integrated environmental sensors attached to a harness on the outside of the firefighter’s 165 protective clothing. Though the portable hub 175 is fixed with a harness, it can be attached to the firefighter’s equipment differently, such as on the belt, the backpack, or on pockets. FIG. 6 illustrates a firefighting team that includes four firefighters 165 and a supervisor 180 using an exemplary system and methods. FIG. 7 illustrates an endurance runner equipped with a wearable device 210 and a portable hub 220.

The central hub and/or portable hub can be arranged to function as a fully capable computing system with one or more processors, memory devices, storage devices, communication interfaces, input/output interfaces, displays, and/or other electronic components arranged to execute the methods described herein. The processor may include one or more microprocessors, controllers, microcontrollers, digital signal processors, application-specific integrated circuits, field-programmable gate arrays, graphics processing units, neural processing units (NPUs), programmable logic devices, or combinations thereof. The memory can include volatile memory, non-volatile memory, cache memory, flash memory, random access memory, read-only memory, solid-state storage, magnetic storage, optical storage, or other computer-readable storage media. The communication interface can support both wired and wireless communication using one or more communication protocols. For example, the communication interface may include Ethernet interfaces, cellular radios, Wi-Fi radios, LoRaWAN or other long range radio communication interfaces, Bluetooth® or ANT+ interfaces, near-field communication interfaces, satellite communication interfaces, serial interfaces, universal serial bus interfaces, or combinations thereof.

As noted above, environmental devices and sensors can be included in the central hub based on the application of the system, where these devices and sensors can be in communication with the central hub through wireless or wired communication. User interfaces can include one or more displays, touchscreens, keyboards, pointing devices, microphones, speakers, or other input/output devices.

As illustrated in FIG. 2, the central hub 120 can include a robust outer protective case to safeguard the internal components for harsh or rugged conditions. FIG. 3 schematically illustrates another embodiment of a central hub 130 that includes an antenna 140 to support more efficient and longer-range data transmission. Although a single antenna 140 is illustrated, the central hub 130 can include multiple antennas configured to support communication redundancy, mesh networking, and communication across multiple protocols or frequency bands, and/or simultaneous communication with multiple devices or hubs. It will be appreciated that a central hub can be selected for a system based on the needs of the team such as number of individuals monitored, the size of the area occupied by the individuals, and the like.

FIG. 4 illustrates an individual 150 with a wearable device 110 and a portable hub 160. The portable hub 160 can be arranged to replace the central hub or supplement the central hub. In replacing the central hub, the portable hub 160 can be arranged to receive data from the wearable device 110 and include algorithms and other software to analyze the data, make determinations and recommendations, and provide such determinations and recommendations to the individual. In supplementing the central hub, the portable hub 160 can be arranged to receive data from the wearable device 110 and transmit that data to the central hub. The portable hub 160 can include algorithms and other software to analyze the data and perform other useful functions but leave certain functions to the central hub.

One example of an implementation of a system is for monitoring a team that is fighting a wildfire. This activity includes a large team of professional firefighters working in extreme conditions in remote areas that requires high coordination and each individual performing his or her task successfully. In this example, each firefighter is equipped with one or more devices that measure biological conditions such as body temperature, blood oxygen saturation level, heart rate, HRV, and cognitive alertness. With reference to FIGS. 5 and 6, it will be appreciated that when fighting a wildfire, the environmental conditions proximate to the firefighter 165 are of paramount importance; thus, attaching the portable hub with integrated environmental device 175 to the firefighter 165 will result in accurate and contextually relevant measurements. It will be understood that under other circumstances, the environmental device can be located generally near the fire independent of the firefighter. The environmental device can be arranged to measure ambient temperature, humidity, particulate matter levels, general oxygen levels, and level of smoke, carbon monoxide, other contaminants in the air, and the like.

FIG. 6 schematically illustrates a system deployed for monitoring a team of firefighters 150. The system includes four firefighters 165 deployed in the field, each equipped with a wearable device 170 and a portable hub 175 with integrated environmental devices. The system further includes a central hub 130 positioned remotely from the fire and equipped with an antenna 140 to facilitate better data transmission. As illustrated in FIG. 6, a supervisor 180 is located near the central hub 130. The system is arranged to monitor the measurements of each firefighter 165 and the surrounding environment and make real-time determinations as to whether each firefighter 165 and/or the team generally is in physical danger or if the physiological and cognitive conditions of the firefighter 165 and environmental conditions allow the team to continue effectively fighting the wildfire. This determination can be provided as feedback directly to the specific firefighter 165 or generally to the team through an indicator on the portable hub 175. Additionally, or alternatively, the determination can be provided to the team supervisor 180 to take appropriate action. In one embodiment, the system is arranged so that the central hub 130 transits information regarding the condition and recommendations for each team member to an application or website accessible to the supervisor 180. As illustrated in FIG. 6, such information can be viewable on a mobile device such as a tablet 190. The condition and recommendations for each team member can be displayed on a dashboard that the supervisor 180 can efficiently and quickly review and take any actions necessary.

Generally, the system is highly configurable and can be arranged to accommodate a number of circumstances and scenarios. In one embodiment useful in evaluating current performance and improving future performance, the system is arranged to gather a high volume of data during an activity and then facilitate post-hoc analysis of the data by the individual or a person responsible for the individual such as a work supervisor or athletic coach. This embodiment is useful for training and monitoring professional endeavors and for athletes seeking to improve performance. The embodiment includes one or more wearable devices, one or more environmental devices, one or more personal hubs, a central hub, and optionally, algorithms, software, and/or other functionality on a remote computing device such as one or more cloud-based or data center servers. In one example, the individual can be equipped with one or more wearable devices that measure heart rate, HRV, body temperature, blood pressure, blood oxygen saturation level, glucose, brain activity, motion of the individual (with a 6-axis motion capable device), and other such measurable physiological and cognitive conditions of the individual. Environmental devices, either incorporated into the central hub or portable hub or positioned in the environment surrounding the individual, can be arranged to measure temperature, humidity, ambient light, pressure, altitude, noise levels, air quality, wind speed, and other such measurable environmental conditions. The physiological, cognitive, and environmental measurements are transmitted to the portable hub or central hub as the data is generated.

The central hub can include functionality to analyze the data and generate reports and charts based on the data. Such reports and charts can be automatically generated by the algorithms and other software of the central hub or can be generated in response to queries. Thus, after the activity, the individual, supervisor, coach or other person can review reports and charts automatically generated by the central hub and/or query the central hub for desired reports or charts summarizing certain aspects of the individual’s performance. The central hub can include an operating system and a number of input and output devices to allow interaction with the central hub. For instance, the central hub can include a display monitor, keyboard, and mouse. In another embodiment, the hub can be arranged to communicate with a client or user interface accessible via a website, browser-based application, and/or mobile application. In such an embodiment, a user can review standard reports and charts and query additional information using a web browser or mobile application on a personal computer, mobile phone, tablet, and other such computing devices.

In another embodiment, the central hub can gather all data generated by the wearable devices and environmental devices and subsequently or in real-time upload the data to a cloud based or other remote server for analysis. The remote server and the algorithms and other software located on the server is included in the systems and methods described herein. Similar to the above description, the individual or other person can interact with the system through a website, browser based application, and/or mobile application via a personal computing device to review standard reports and charts and query the system for customized reports and charts. In certain embodiments, access to the central hub and associated data can occur through a local wireless network or hotspot generated by the central hub, through an existing local network, and/or through secure remote networking technologies enabling remote visualization and interaction with the system.

The above description of the systems and methods and the particular arrangements of the central hub, algorithms and software, and the use of remote servers can be applied to all embodiment of systems and methods disclosed herein.

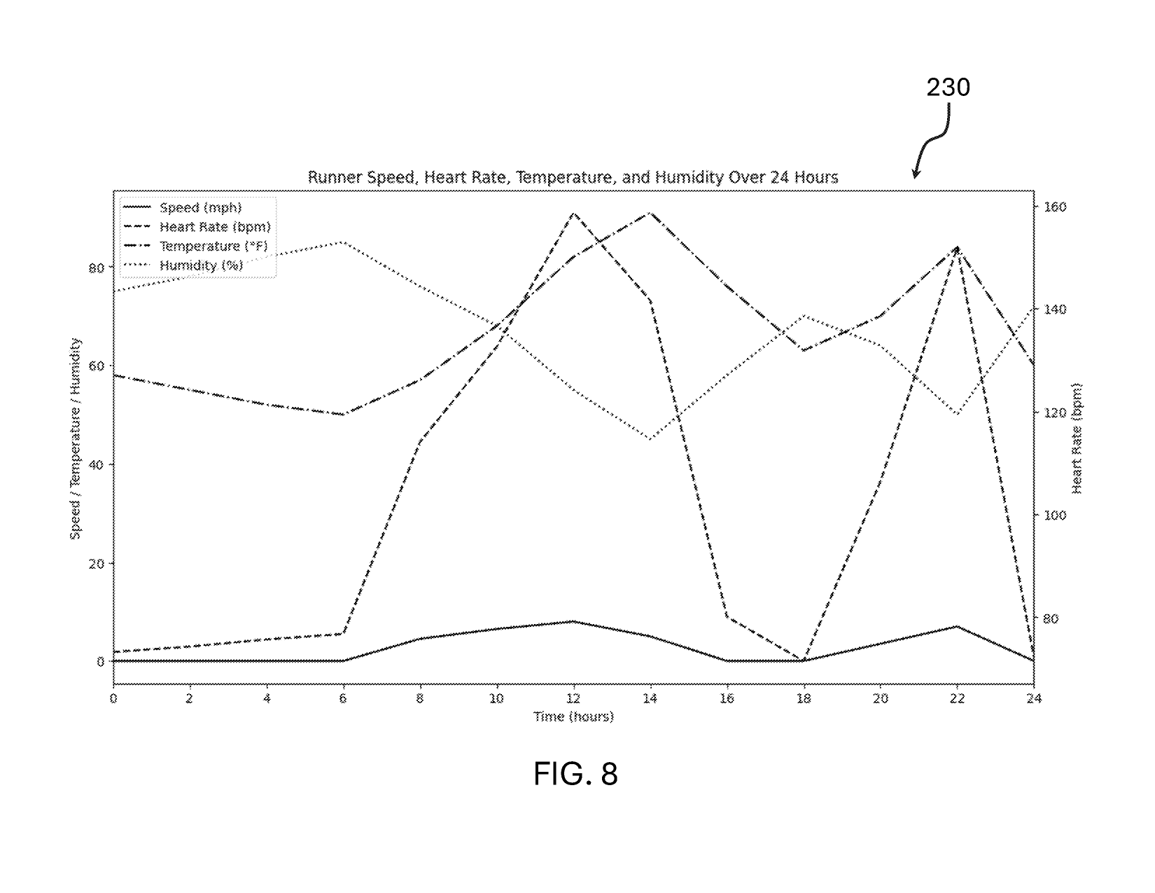

FIGS. 7 and 8 illustrate one exemplary use of the systems and methods disclosed herein. FIG. 7 illustrates a runner 200 equipped with a wearable device 210 arranged to measure heart rate, heart rate variability, blood oxygen saturation levels, perspiration rate, and movement of the runner 200. The runner 200 is further equipped with a portable hub 220 including integrated environmental sensing devices arranged to measure ambient temperature, humidity, and other environmental conditions proximate to the runner 200. The portable hub 220 is configured to receive physiological data from the wearable device 210 and can further provide local feedback, alerts, recommendations, and visualizations directly to the runner 200 during the activity. In this example, the runner 200 is an endurance athlete that competes in races that last many hours and span many miles. During a race or training session that lasts 24 hours, the system gathers the physiological and environmental data over that b hour period. In certain embodiments, critical physiological, environmental, operational, and/or alert data can be transmitted during the race or training session from the portable hub 220 to a central hub located nearby coaches, race organizers, medical personnel, or other remote operators using direct or meshed radio-frequency communication networks, and/or cellular, 4G, Wi-Fi, or satellite communication systems when available. At the conclusion of the race or training session, the system generates one or more standard reports such as the report 230 illustrated in FIG. 8. The report 230 charts the runner’s speed and heart rate and the ambient temperature and humidity over the 24 hours session. After the session, the runner, the runner’s coach, or another person can review the report 230 to help evaluate the runner’s performance and develop strategies and tactics for improving the runner’s future performance.

The system can include additional features such as direct feedback from the runner regarding the runner’s perceived state of physical and mental condition. This feedback can be gathered through the portable hub 220 where the runner can efficiently and effectively periodically enter data such as through a series of buttons or a touch screen. In another example, the runner can be in verbal communication with his coach or training team through a cell phone enabled earpiece or another such device and relay the runner’s perceived state of physical and mental condition to the training team, and the training team can manually enter that data into the system via the central hub interface.

As noted, the individual, coach, or other person can also query the system for customized reports and charts. For example, it may be instructive to additionally overlay the feedback from the runner onto the chart in FIG. 8 so that the runner’s perceived physical and mental condition can be taken into account to understand the runner’s speed and heart rate under the environmental conditions during the race or training session.

In this example, as well as in other examples, the central hub and portable hub can be arranged to store data locally during a session. As will be appreciated, when engaging in an activity that potentially spans a large geographic area, it may be more practical to store data locally on portable hubs and subsequently download the data to the central hub or to a remote server. Such arrangements are applicable to methods that include post-hoc analysis and review. However, the system can also be arranged to transmit data from portable hubs to the central hub or a remote server over large distances to accommodate post-hoc as well as real-time analysis and review.

While the above example and its variations use an endurance athlete as the subject individual, it will be appreciated that such systems and methods can be used for a wide variety of professional, athletic, and recreational activities. To name just a few examples, the systems and methods are applicable to doctors and other medical personnel working long, grueling hours where maintaining mental alertness and judgment is paramount; workers performing in dangerous and hazardous conditions such as coal miners, firefighters, and military personnel; extreme recreational activities such as mountaineering; and athletic activities such as competitive sailing, cycling, running, or collegiate sports.

In another embodiment useful in monitoring current performance and providing real-time feedback to individuals or on-site supervisors or coaches, the central hub is arranged to automatically and continuously gather and aggregate data from one or more wearable devices and integrated or independent environmental devices, perform analytics locally, and provide real-time individual and team-level feedback, including optionally via a digital display, of conditions and recommendations to assist decision-makers in guiding the individual or team. The central hub includes algorithms and other software to perform analytics on the data locally and provide real-time feedback and recommendations for individuals or at the team-level. Such feedback and recommendations can be provided via a digital display, whether on a monitor associated with the central hub or via a website, browser-based application, and/or mobile application accessible through a local wireless network, hotspot, existing network connection, or secure remote networking connection using a personal computing device. Such real-time feedback and recommendations provide actionable direction for decision-making.

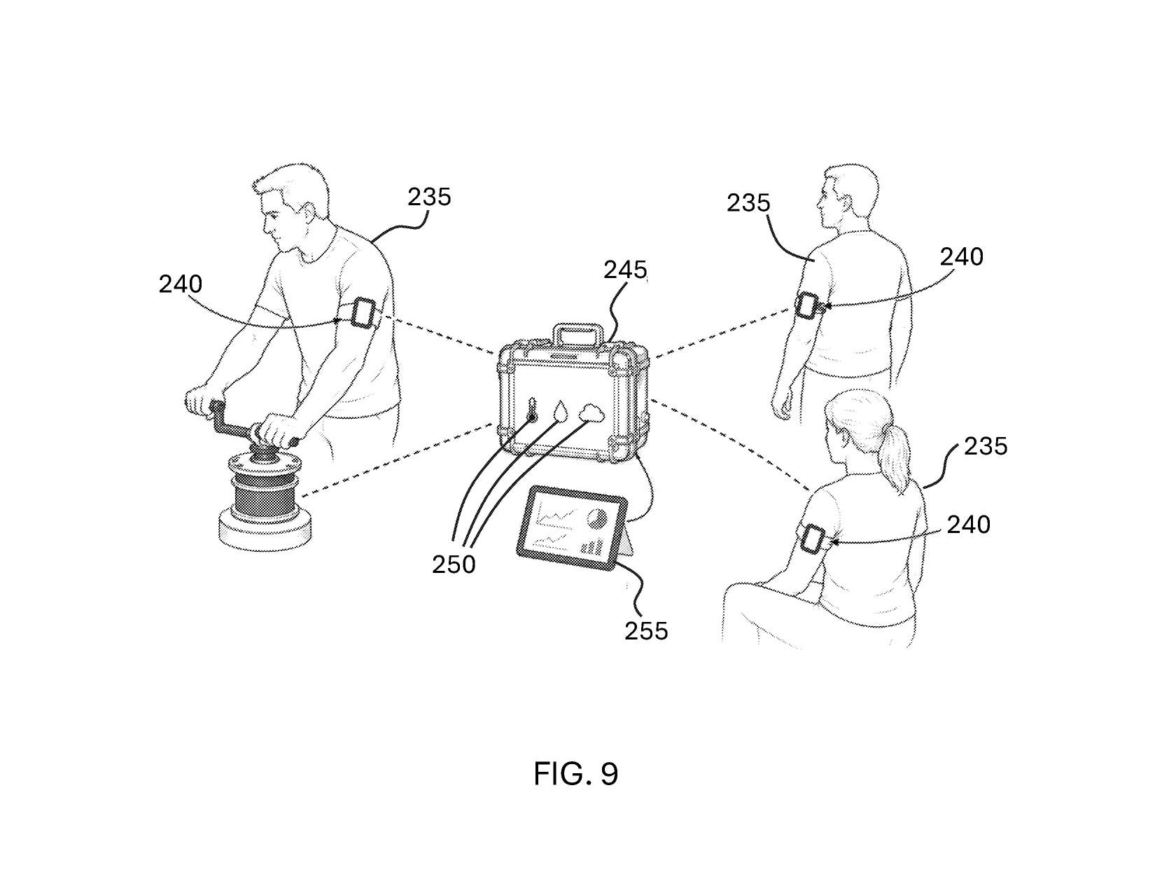

FIG. 9 schematically illustrates another embodiment of the system in which individuals 235 are equipped with one or more wearable devices 240, where the wearable devices 240 are arranged to directly communicate with a central hub 245. The central hub 245 can include a number of integrated environmental sensors 250. In this embodiment, the wearable devices 240 can directly transmit data to the central hub 245 using short-range wireless communication protocols such as Bluetooth® and/or ANT+ while remaining within communication range of the central hub 245. Such an arrangement can be advantageous in operational environments including sailboats, motor vehicles, aircraft, medical facilities, stadium sports, and other environments in which monitored individuals remain in relatively close proximity to the central hub 245. The central hub 245 can aggregate physiological and environmental data, perform local analytics on the data, generate feedback and recommendations, and optionally communicate with remote systems, cloud-based platforms, or remote personnel. The system can further include a local display device 255 to display conditions, feedback, recommendations, and the like to the individuals 235.

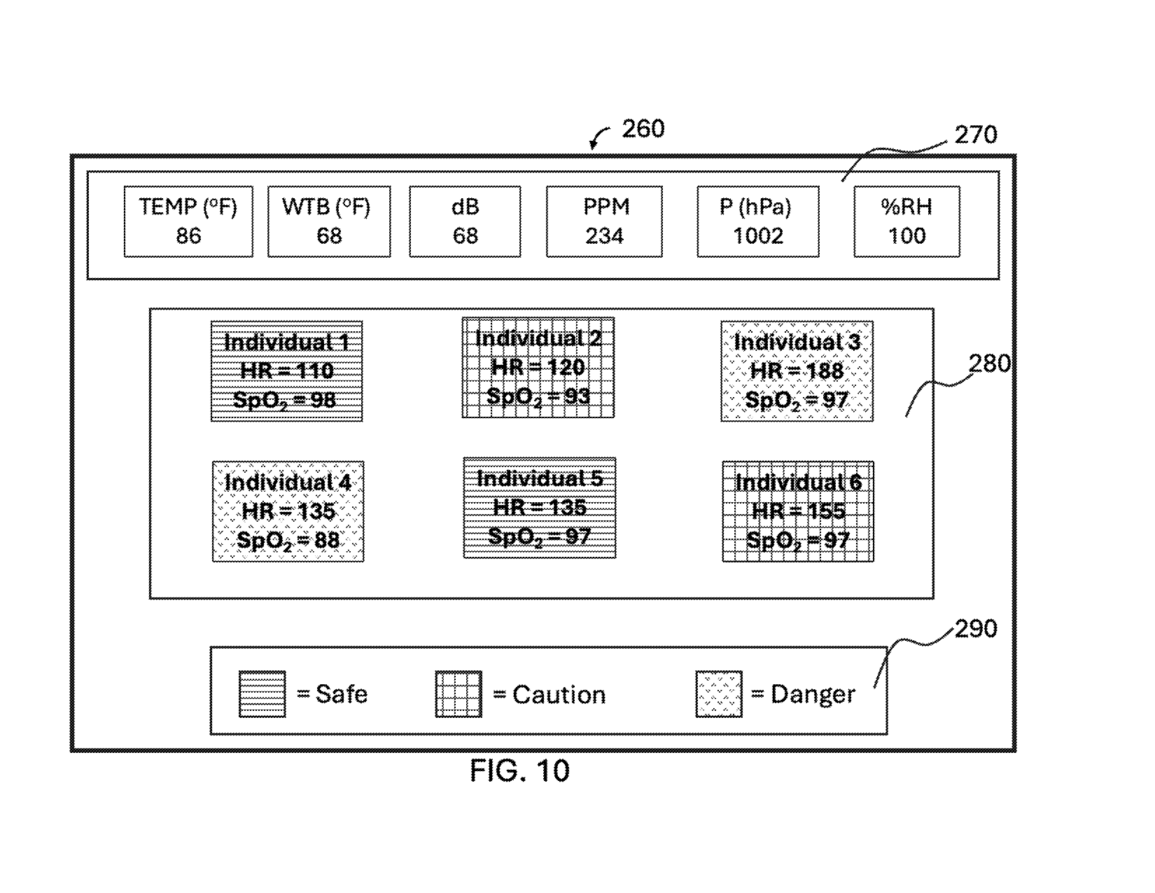

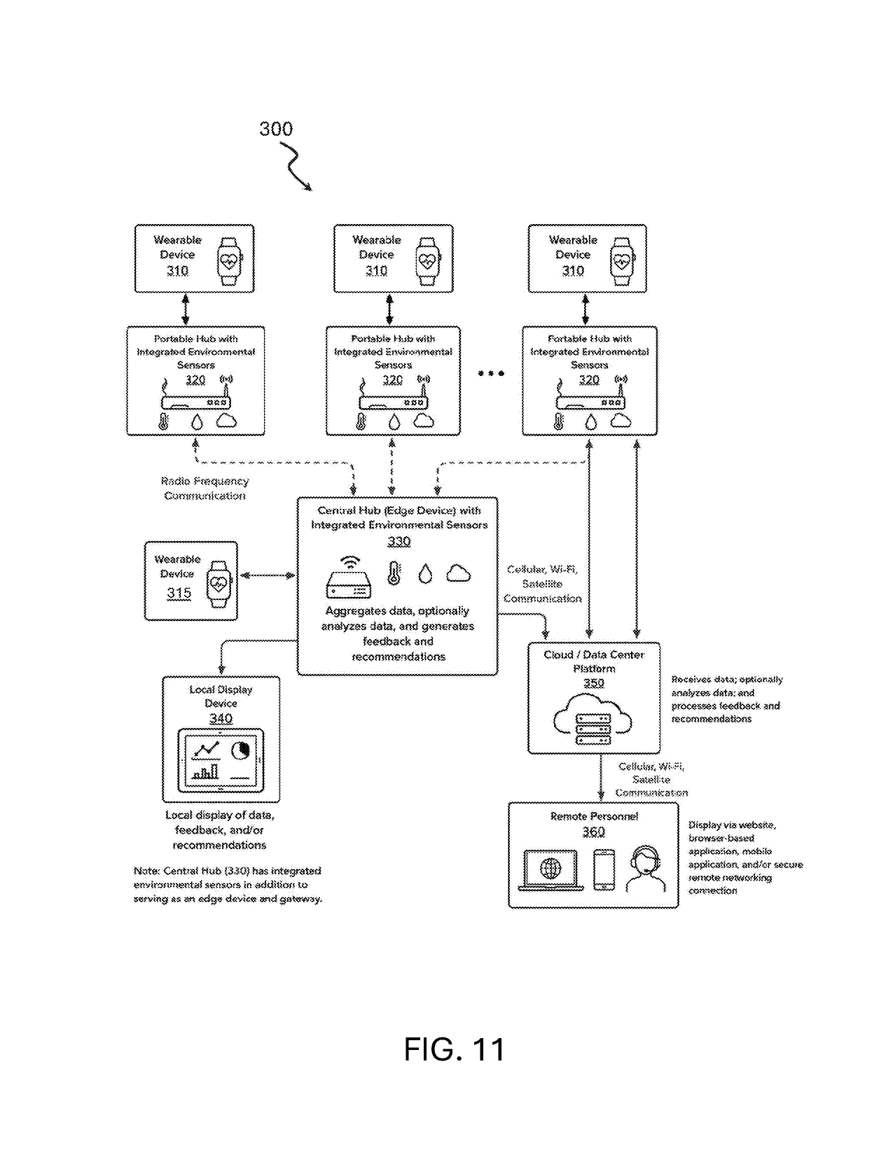

As illustrated in FIG. 10, the system can be arranged to provide real-time feedback and actionable information using a dashboard 260 that can be rendered on a visual display. The dashboard 260 includes a section 270 that displays environmental conditions. In this example, the environmental conditions displayed include ambient temperature, wet bulb temperature (relevant to heat stress and safety), noise levels, particulate count of a substance, atmospheric pressure, and relative humidity. The dashboard also includes a section 280 that displays physiological conditions of individuals and general concern level for the individuals. In this example, six individuals are monitored with the individual’s heart rates and blood oxygen saturation levels separately displayed in individualized boxes. Additionally, the background of each box is shaded to indicate the level of concern for the individual. The dashboard 260 further includes a legend 290 that indicates what background shading equates to the various levels of concern for the individuals. In this example, there are three levels of concern – “safe,” which means no concern; “caution,” which means one or more physiological or environmental condition is in a range that may cause harm to the individual; and “danger,” which means one or more physiological or environmental conditions is in a range that will cause harm to the individual.

The determination of level of concern is based on set ranges for the various physiological and environmental conditions, which can be configured pre-hoc by a medical team, the team lead, or coaches. In this example, the ranges for an individual’s heart rate are: (i) under 150 beats per minute, safe; (ii) between 150 and 180 beats per minute, caution; and (iii) over 180 beats per minute, danger. The ranges for an individual’s blood oxygen saturation level are: (i) 95.00% to 100%, safe; (ii) between 90.00 and 94.99%, caution; and (iii) under 90%, danger. Ranges for wet bulb temperature are: (i) under 80° F., safe; (ii) between 80° F. and 88° F., caution; and (iii) above 88° F., danger.

In comparing these ranges to the conditions as displayed on the dashboard 240, the wet bulb temperature is 68° F., which means all individuals can safely continue on with the activity. However, if the wet bulb temperature were to rise to 80° F. and 88° F., then each individual would be considered in the cautious or dangerous range, respectively. For individuals 1 and 5, heart rates and blood oxygen saturation levels are in the safe range; thus, the individualized boxes are shaded to represent the individuals are safe. For individuals 2 and 6, individual 2 has a blood oxygen saturation rate that is 93% and individual 6 has a heart rate that is 155, both in the cautious range; thus, the individualized boxes are shaded to represent the individuals should be cautious in continuing the activity. For individuals 3 and 4, individual 3 has heart rate of 188 and individual 4 has a blood oxygen saturation rate that is 88%, both in the dangerous range; thus, the individualized boxes are shaded to represent the individuals are in danger if continuing the activity.

This arrangement of the dashboard provides actionable information for the individual, supervisor, coach, or other person responsible for the individuals. For example, for individuals 1 and 5, a supervisor or coach may instruct those individuals to continue on with the current activity or instruct those individuals that they may increase the intensity of the activity. For individuals 2 and 5, a supervisor or coach may inform the individuals of the concern and continue to closely monitor the applicable conditions for the individuals, may instruct the individuals to decrease the intensity of the activity, or may instruct the individuals to take a break from or cease the activity. For individuals 3 and 4, a supervisor or coach may instruct the individuals to take an immediate break from or cease the activity and may additionally instruct the individual to seek medical attention.

The dashboard 240 of FIG. 9 is but one example of an arrangement of information and indicators. It will be appreciated that the dashboard can be configured in a variety of ways that inform and warn individuals, supervisors, coaches, and other concerned persons about the condition of individuals. In other examples, colors (such as green, orange, and red) can be used to illustrate concern; audible alerts can be used to make persons aware of a change in concern level for an individual; and the information displayed can explicitly provide a recommendation such as, “stop activity immediately” or “reduce intensity of activity.” In certain embodiments, the dashboard can further display proprietary indexes, scores, risk levels, or other derived indicators generated from combinations of physiological, environmental, operational, historical, and/or behavioral metrics measured by the system.

In another embodiment useful in monitoring current performance and providing real-time feedback to remote supervisors, coaches, and other concerned persons, the system is arranged to automatically and continuously gather and aggregate data from one or more wearable devices and environmental sensors. The data can be analyzed locally on the central hub or portable hub or with the use of connectivity (i.e., internet connectivity WiFi, cellular, satellite, and the like). The data can be forwarded onto a remote cloud based or data center server for analysis, and with such connectivity, the conditions and recommendations can be provided to a web dashboard or mobile application as described above for review by remote supervisors. This arrangement provides for real-time monitoring of remote teams such as medical doctors, sports teams, and audiences for entertainment.

In another embodiment, the system can be arranged such that the wearable devices transmit data to a portable hub wirelessly, such that the portable hub receives data from integrated or independent environmental sensors, and that the portable hub transmits data over long distances to either a central hub or a cloud based or data center based server or application. Such an arrangement is useful for teams deployed in remote environments. One example of such teams are military deployments and search and rescue teams. The individual is equipped with both wearable devices and a portable hub which includes the functionality to stream data over large distances. Such a system provides real-time and actionable information to team leaders and other concerned persons.

FIG. 10 schematically illustrates a system diagram to provide an overview of an exemplary system 300. In this example, the system 300 includes three wearable devices 310 associated with three portable hubs 320, where each portable hub 320 includes integrated environmental sensors. The wearable devices 310 and portable hubs 320 can be paired up and worn by an individual. Each of the wearable devices 310 is arranged to communicate and transmit data to a portable hub 320, which in turn communicates to a central hub 330. The communication between the portable hubs 320 and central hub 330 can be facilitated through radio frequency communications. The system 300 includes an additional wearable device 315 arranged to communicate directly with the central hub 330. The central hub 330 can be an edge device and serve as a gateway for the aggregation of data and optionally analyze data and generation of feedback and recommendations. The communication between the wearable devices 310 and the portable hubs 320 is illustrated as two-way communication. However, in certain embodiments, the communication can be one-way.

The central hub 330 can be further arranged to locally display the data, feedback, and/or recommendations on a display device 340 such as a tablet. Alternatively, or additionally, the central hub 330 can be arranged to transmit data, feedback, and/or recommendations to a cloud-based or data center-based platform 350 through cellular, Wi-Fi, satellite, radiofrequency, or other communication systems. The platform 350 can be arranged to receive the data and optionally analyze the data, as well as the feedback and recommendations. The platform 350 can be arranged to facilitate the display of the data, feedback, and/or recommendations to remote personnel 360 through a website, browser-based application, mobile application, and/or secure remote networking connection.

In one embodiment, the central hub (serving as a gateway device) can include a Raspberry Pi 5 with solid state drive SSD storage running containerized microservices. The central hub, wearable devices, and portable hub with integrated environmental sensors can be arranged to communicate via wireless communication protocols such as Bluetooth® low energy (BLE), ANT+, long range radio communications, and other such protocols and technologies. The central hub can be arranged to concurrently manage the connections to all wearable and portable hubs using a dual-adapter architecture to ensure scan reliability.

The foregoing description and figures are provided for purposes of description and illustration and are not intended to be exhaustive or to limit the disclosure in any way including not limiting the disclosure to the precise forms and arrangements disclosed. Modifications and variations are possible in light of the above teachings. The embodiments were chosen and described to explain principles of the disclosure and practical applications thereof to enable others skilled in the art to utilize the disclosure in various embodiments and with various modifications as suited to the particular use contemplated.

We claim:

1. A system for monitoring, determining, and predicting the condition of one or more individuals and providing one or more recommendations to the one or more individuals, the system comprising:
one or more wearable devices secured to each individual, wherein each wearable device is arranged to gather one or more data points regarding at least one physiological or cognitive aspect of the individual;
one or more environmental devices positioned in the environment surrounding each individual, wherein each environmental device is arranged to gather one or more data points regarding at least one aspect of the environment surrounding the individual; and
one or more hubs.

2. The system of claim 1, wherein the one or more hubs is a central hub spaced away from the one or more individuals and includes at least one algorithm arranged to analyze the gathered one or more data points.

3. The system of claim 2, wherein each of the one or more wearable devices and one or more environmental devices is arranged to transmit the one or more data points to the central hub.

4. The system of claim 3, wherein the algorithm includes functionality to analyze the gathered one or more data points and quantify at least one aspect of the one or more individuals’ physiological or cognitive condition.

5. The system of claim 4, wherein the algorithm includes functionality to analyze the gathered one or more data points and quantify at least one aspect of the environmental conditions.

6. The system of claim 5, wherein the algorithm includes functionality to analyze the quantified at least one aspect of the one or more individuals’ physiological or cognitive condition and at least one aspect of the environmental conditions and determine a recommendation for each individual.

7. The system of claim claim 6, wherein the central hub is arranged to transmit the recommendation for each individual to the wearable device secured to the individual.

8. The system of claim 7, wherein the system includes access to data that is external to the system.

9. The system of claim 8, wherein the external data includes weather forecasts for the environment surrounding each individual.

10. The system of claim 9, wherein the algorithm includes functionality to analyze the weather forecast and predict a future physiological or cognitive condition for each individual.

11. The system of claim 10, further comprising a video display arranged to visually display the gathered one or more data points, the quantified at least one aspect of the one or more individuals’ physiological or cognitive condition, the quantified at least one aspect of the environmental conditions, the prediction of the future physiological or cognitive condition for each individual, and the recommendation for each individual.

12. The system of claim 11, wherein the visual display is arranged as a dashboard and organized to group all information about each of the one or more individuals with an identifier for the individual.

13. The system of claim 12, wherein the dashboard includes a visual indicator to identify the quantified at least one aspect of the one or more individuals’ physiological or cognitive condition, the prediction of the future physiological or cognitive condition for each individual, or the recommendation for each individual.

14. The system of claim 10, further comprising a database arranged to store the gathered one or more data points, the quantified at least one aspect of the one or more individuals’ physiological or cognitive condition, the quantified at least one aspect of the environmental conditions, the prediction of the future physiological or cognitive condition for each individual, and the recommendation for each individual.

15. The system of claim 1, wherein the one or more data points regarding at least one physiological or cognitive aspect of the individual include at least one of: heart rate, beat-to-beat intervals and derived metrics, blood oxygen saturation levels, body temperature, respiration rate, hydration level, blood pressure, perspiration rate, blood glucose, blood pressure, electrical activity of the brain, and cognitive alertness.

16. The system of claim 1, wherein the one or more data points regarding the regarding at least one aspect of the environment surrounding the individual includes at least one of: ambient temperature, humidity, particulate matter level, oxygen level, smoke level, carbon monoxide level, ambient light, air pressure, altitude, noise level, air quality, and windspeed.

17. The system of claim 8, wherein data that is external to the system includes at least one of: wind direction, wind speed, anticipated temperature, rainfall, and humidity, windspeed, air pressure, vehicle information such as speed, direction, motion, sunrise time, and sunset time.

18. The system of claim 2, wherein the at least one hub further includes one or more portable hubs with at least one portable hub secured to each individual.

19. The system of claim 18, wherein each wearable device and each environmental device is arranged to transmit data to one of the portable hubs.

20. The system of claim 19, wherein each of the one or more portable hubs includes at least one algorithm arranged to analyze the gathered one or more data points.

21. The system of claim 18, wherein each of the environmental devices is integrated with one of the portable hubs.

22. The system of claim 18, wherein each portable hub is arranged to transmit data to the central hub.
